(19) United States
(12) Patent Application Publication
(10) Pub. No.: US 2022/0362071 A1
(43) Pub. Date: Nov. 17, 2022

(54) Absorbent Underwear Insert

(71) Applicant: Mesue Ebontene, Glen Burnie, MD (US)

(72) Inventor: Mesue Ebontene, Glen Burnie, MD (US)

(21) Appl. No.: 17/713,377

(22) Filed: Apr. 5, 2022

Related U.S. Application Data
(60) Provisional application No. 63/188,083, filed on May 13, 2021.

(51) Int. Cl. A61F 13/505; A61F 13/62

(52) U.S. Cl. CPC A61F 13/505; A61F 13/62; A61F 2013/15008

(57) ABSTRACT

The present invention relates to an absorbent insert for underwear. The absorbent insert is removably-secured to an inner surface of a waistband of a user's underwear using integrated adhesives such as hook and loop fasteners. The insert has a fabric body, having an outer surface and an inner surface with a two-layer absorbent pad. The absorbent pad has an impervious layer and a thicker absorbent layer. The absorbent layer covers the crotch region of a user to absorb moisture, urine and sweat without staining the underwear or any clothing. The absorbent insert is machine washable and reusable, and comes in various sizes, shapes and colors to fit a user's underwear.

### CROSS-REFERENCE TO RELATED APPLICATION

The present application claims priority to, and the benefit of, U.S. Provisional Application No. 63/188,083, which was filed on May 13, 2021 and is incorporated herein by reference in its entirety.

### FIELD OF THE INVENTION

The present invention relates generally to the field of absorbent pads. More specifically, the present invention relates to a urine absorbing insert or attachment made up of absorbent material and designed to be attached on the inside of a user's underwear. The urine absorbing attachment can be easily placed within a user's undergarments and secured in place via hook and loop fasteners or adhesives. The urine absorbing attachment when placed inside a user's underwear, absorbs moisture and urine leaks, and therefore prevents unwanted, embarrassing stains on clothing. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, many users face problems with fully emptying their bladders due to various health complications. Some users also suffer from urine incontinence of loss of bladder control. Urine incontinence is not only a medical problem that causes uncontrollable urine leaking, but can also affect a user emotionally and psychologically. Users suffering from urine incontinence lose their confidence and are unable to participate in their normal daily activities. By way of statistics, a quarter to a third of men and women in the U.S. suffer from urinary incontinence, due to aging and other health-related conditions.

Users who are unable to fully empty their bladder due to health complications or general incontinence may experience urine leaking into their undergarments and down their thighs and/or legs. Leaking urine through their undergarments can cause skin rashes and/or irritation, along with unwanted odors that require constant undergarment changes. Urine leakage can also cause social embarrassment among users. Users with urine incontinence issues may be reluctant to attend social gatherings/interactions. Such issues, if not resolved, can negatively impact a user's quality of life.

Therefore, there exists a long felt need in the art for a personal care article that can be used to overcome the consequences related to urine incontinence. There is also a long felt need in the art for a personal care article that is designed to be used by both men and women. Additionally, there is a long felt need in the art for a personal care article that can be easily attached to a standard pair of underwear. Moreover, there is a long felt need in the art for a personal care article that absorbs the urine leaked inadvertently by people suffering from urine incontinence or other medical problems, and prevents urine from leaking in undergarments, and down thighs and legs. Further, there is a long felt need in the art for a personal care article that absorbs leaking urine and prevents skin rashes and other irritation among users with general incontinency issues. Furthermore, there is a long felt need in the art for a personal care article that prevents urine leaking, and therefore prevents unwanted odors. There is a long felt need in the art for a personal care article that eliminates the need to frequently change undergarments due to urine leakage. There is a long felt need in the art for a personal care article that prevents urine from dripping through a user's underwear and causing wet clothes. There is a long felt need in the art for a personal care article that enables users with incontinency and other similar health issues to confidently attend social gatherings/interactions, without worrying about inadvertent urine leakage. Finally, there is a long felt need in the art for a personal care article that prevents unwanted and embarrassing stains on clothing due to urine leakage, and allows users to wear normal undergarments without worry of urine leakage.

The subject matter disclosed and claimed herein, in one embodiment thereof, comprises an absorbent underwear insert that is configured to attach within a user's underwear for absorbing urine leaks, sweat and/or moisture, and prevents staining of a user's underwear and clothing due to bladder complications and incontinency problems. The absorbent insert comprises a fabric body having an outer surface and an inner surface, wherein the fabric body has convexly opposing sides, an absorbent pad with a thick absorbent layer and a thin impervious layer sewn to the inner surface of the fabric body. The thick absorbent layer is positioned to cover the crotch region of a user to absorb urine leaks, sweat and/or moisture. Additionally, hook and loop, Velcro®® or other adhering fasteners are positioned on an outer surface of the underwear insert to attach the insert to the waistband of a user's underwear. The absorbent layer is manufactured of an absorbent material such as cotton or synthetic polymer, and the fabric body is manufactured of cotton or spandex. The absorbent insert is washable and reusable for the convenience of users.

In this manner, the novel absorbent insert for underwear of the present invention accomplishes all of the forgoing objectives, and provides a relatively safe, easy and convenient solution for users suffering with incontinency and other similar health issues. Specifically, the absorbent insert for underwear prevents urine from leaking through a user's undergarments, and prevents clothes from unwanted and embarrassing stains. The absorbent insert for underwear of the present invention is also user-friendly, and can be easily attached and secured inside any conventional underwear, and its absorbent material absorbs moisture, sweat and/or urine leaks. The absorbent insert for underwear enables users to confidently attend social gatherings/interactions without worrying about inadvertent urine leaks. Additionally, the absorbent insert for underwear is reusable, and can be easily detached from a user's underwear for cleaning, or can be cleaned while attached to a user's underwear as well.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an absorbent underwear insert device. The absorbent underwear insert device is configured to attach within a user's underwear for absorbing urine leaks, sweat and/or moisture, and prevents stains due to bladder complications and/or incontinency problems. The absorbent insert device further comprises a fabric body having an outer surface and an inner surface. The fabric body comprises convexly opposing sides, an absorbent pad having a thick absorbent layer and a thin impervious layer sewn to the inner surface of the fabric body. The thick absorbent layer is positioned to cover the crotch region of a user to absorb urine leaks, and hook and loop or Velcro®® fasteners positioned on an outer surface attach to the waistband of a user's underwear to removably secure the absorbent insert within a user's underwear. The thick absorbent layer is manufactured of an absorbent material, such as cotton or synthetic polymer and the fabric body is manufactured of cotton or spandex.

In yet another embodiment of the present invention, the absorbent insert device is foldable along a bottom central axis. Further, the absorbent insert device is positioned between a user's thighs, with a front side covering the crotch area and the back side covering the buttocks (or glute) area of a user.

In yet another embodiment of the present invention, an absorbent garment device that conforms to the shape of a user's underwear is disclosed. The absorbent garment device is attached along a waistband of a user's undergarment, using adhesive fasteners such that the garment remains attached to the underwear during use and does not have to be worn separately. The absorbent garment device is machine washable and reusable, and can be washed separately or washed while attached to a user's underwear.

In a further embodiment of the present invention, an absorbent insert device for a user's underwear is disclosed. The absorbent insert device includes a fabric body having convexly opposing sides to conform to the shape of a user's underwear, adhesive fasteners for securing to a waistband of the user's underwear, an impervious layer sewn to a portion of the fabric body and a thick absorbent layer sewn to said impervious layer, which is configured to absorb urine leaks and moisture.

In yet another embodiment, the thick absorbent layer is wide enough to cover the crotch area of a user, and conforms to the shape of the crotch covering area of a user's underwear. The absorbent insert is manually attached to a user's underwear and positioned between a user's body and their underwear to absorb any discharged urine or moisture, and can then be discarded.

In yet another embodiment of the present invention, a urine-absorbent insert device for a user's underwear is disclosed. The insert device is configured to attach to a user's underwear and is positioned between a user's body and his or her underwear. The insert device is positioned between a user's thighs, and covers the crotch area of a user to absorb discharged urine and moisture and to prevent staining. An absorbent pad having an impervious layer and an absorbent layer absorbs the urine and moisture.

In yet a further embodiment of the present invention, a method of absorbing discharged urine due to bladder and incontinency issues via an underwear insert device is disclosed. The method includes initially providing an elongated absorbent pad having an absorbent layer and an impervious layer, attaching the insert to an inner surface of a user's waistband using adhesive fasteners disposed on the outer surface of the insert, wearing the underwear with the attached insert such that the absorbent pad covers the crotch area of a user and removing the insert from the underwear for washing and re-use.

The color and shape of the absorbent insert device of the present invention is based on the shape and size of a user's underwear to which the insert device is configured to attach. The absorbent insert device can be integrated within any type, size or design of underwear and/or shorts. The insert device can be manufactured of natural fibers such as cotton, hemp or silk, of regenerated fibers such as rayon or acetate and/or of synthetic fibers such as nylon, acryl, polyester or polyurethane, etc., and mixtures thereof. The absorbent insert device is also smooth against the crotch area of a user, and does not block the leg openings of a user's underwear, thus maintaining the ventilation effect of the underwear.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the absorbent underwear insert device of the present invention in accordance with the disclosed architecture;

FIG. 2 illustrates a perspective view showing the absorbing insert device of the present invention in a flat state in accordance with the disclosed architecture;

FIG. 3 illustrates a sectional view showing the layers of the absorbent pad of the absorbent insert device of the present invention in accordance with the disclosed architecture;

FIG. 4 illustrates a perspective view showing how the absorbent attachment device of the present invention is placed within an undergarment for use in accordance with the disclosed architecture;

FIG. 5 illustrates a front perspective view showing exemplary underwear with an attached absorbent underwear insert device of the present invention in accordance with the disclosed architecture; and

FIG. 6 illustrates a front perspective view showing trunk-shaped underwear with an attached absorbent underwear insert device of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for a personal care article that can be used to overcome the consequences related to urine incontinence. There is also a long felt need in the art for a personal care article that is designed to be used by both men and women. Additionally, there is a long felt need in the art for a personal care article that can be easily attached to standard underwear. Moreover, there is a long felt need in the art for a personal care article that absorbs urine leaks, and prevents urine from dripping in a user's undergarments and down the thighs and legs. Further, there is a long felt need in the art for a personal care article that absorbs urine leaks and prevents skin rashes and/or irritation. Furthermore, there is a long felt need in the art for a personal care article that prevents urine leaks and therefore prevents unwanted odors. There is a long felt need in the art for a personal care article that eliminates the need to frequently change undergarments. There is a long felt need in the art for a personal care article that prevents urine from leaking through a user's underwear and causing wet clothes. There is a long felt need in the art for a personal care article that enables users with incontinency and other similar health issues to confidently attend social gatherings/interactions. Finally, there is a long felt need in the art for a personal care article that prevents unwanted and embarrassing stains on clothing due to urine leaks, and allows users to wear their normal undergarments.

The present invention, in one exemplary embodiment, is a novel absorbent garment device that conforms to the shape of a user's underwear. The absorbent garment device is attached along a waistband of a user's undergarment using adhesive fasteners such that the garment remains attached to a user's underwear during use and does not have to be worn separately. The absorbent garment is machine washable and reusable, and can be washed separately or washed while attached to a user's underwear.

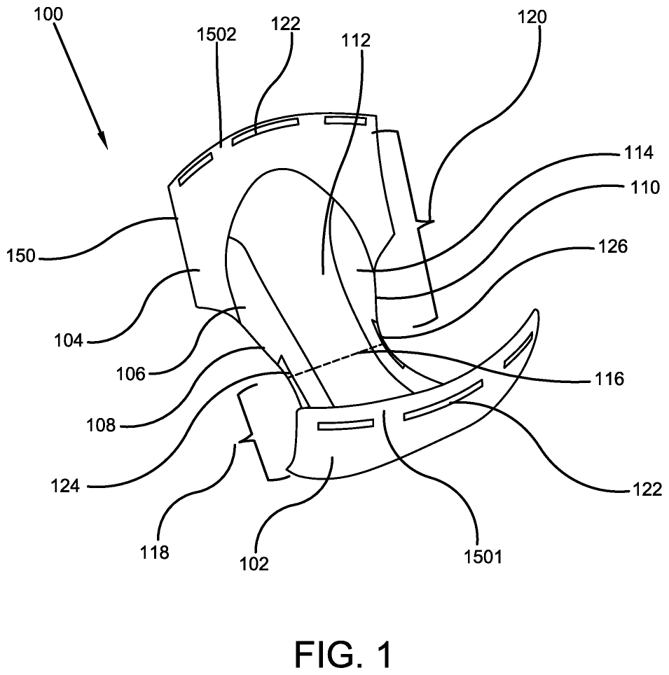

Referring initially to the drawings, FIG. 1 illustrates a perspective view of one embodiment of the absorbent underwear insert device of the present invention. The absorbent underwear insert device 100 is designed to secure within a standard pair of underwear using a soft fastener that allows the insert device 100 to be secured to a waistband of the underwear and conforms to the shape of the underwear. The insert device 100 absorbs urine and moisture to prevent staining of undergarments and clothing. The insert device 100 absorbs sweat and urine in both male and female underwear.

The insert device 100 comprises a fabric body 150 having an outer surface 102 that is configured to adhere to a user's underwear and contacts the underwear surface. The insert device 100 comprises an inner surface 104 that is configured to contact the user during use in a manner similar to when underwear is worn and contacts the body surface. The inner surface 104 has a sewn in or permanently embedded absorbent material pad 106 which covers the crotch region of a user. The insert device 100 also has convexly opposing sides 108, 110 that conform and shape to the crotch area of a user.

The absorbent material pad 106 comprises two layers secured together for effective absorption of urine and sweat and stain prevention. The top layer 112 is positioned to cover the crotch area of a user, and is made up of a thick layer of an absorbent material. The top layer 112 effectively absorbs urine and sweat without providing any discomfort to the user. The bottom layer 114 is made up of a thin layer of an impervious material and eliminates staining of the undergarment and/or clothing.

The insert device 100 is configured to fold along the central bottom fold line 116 that is positioned to align with the central bottom fold line of a user's underwear. When the insert device 100 is worn by either a male or female, the central bottom fold line 116 lies between the legs of a user, and thus provides symmetric coverage for both a front side 118 and a back side 120 of the insert device 100.

The insert device 100 is configured to be removably-secured to an inner surface of a waistband of a user's underwear using hook and loop fasteners or Velcro®, or any other suitable fasteners as is known in the art. Specifically, the outer surface 102 near a first end 1501 and a second end 1502 comprises hook and loop fasteners or Velcro® 122, which act to secure the insert device 100 to the waistband of the underwear. Both the front side 118 and the back side 120 comprise hook and loop fasteners 122 on the outer surface 102 for secure attachment. To provide additional attachment with the underwear, the convexly opposing side 108 has hook and loop fasteners 124, and the second convexly opposing side 110 has a separate hook and loop fastener 126 to attach to the underwear. In one embodiment, the front side 118 and the back side 120 of the insert device 100 can also feature an adhering mechanism on the outer surface 102 for secure attachment to a user's underwear.

It should be appreciated that the dimensions of the absorbent material pad 106 are such that it covers the crotch area of a user. The insert device 100 is machine washable and reusable. The insert device 100 can be washed separately by detaching it from a user's underwear, or can be washed while attached to a user's underwear.

The absorbent material pad 106 provides protection against urine leaks or sweat marks on underwear, jeans, slacks and any other type of clothing. During use, the absorbent material pad 106 is positioned over the crotch region, such that urine leaks are absorbed to prevent staining of the underwear and clothing. The insert device 100 is manufactured of soft fabric that is woven or knitted with elastic, stretchable strands. The insert device 100 can be used with any conventional underwear for both adults and children. Further, the insert device 100 is not visible from the outside, thereby giving a conventional look to the underwear. The absorbent pad 106 is attached to the fabric body 150 by sewn seams. The seam can be a French seam, or other seaming technique that leaves no raw fabric edges exposed, or any other suitable securing means as are known in the art.

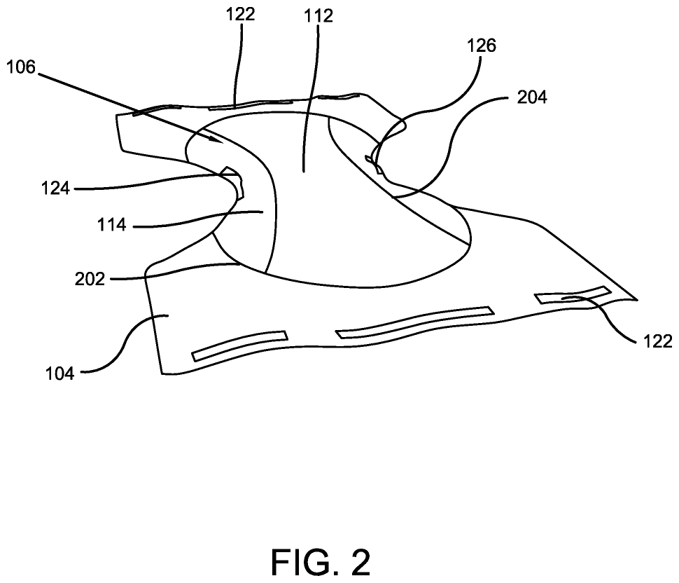

FIG. 2 illustrates a perspective view showing the absorbing insert device of the present invention in a flat state. The absorbing insert device 100 is standalone product that can be removably secured to underwear of any shape or size. The insert device 100 comes in a variety of sizes to accommodate users of varying size, age, and gender. The absorbent pad 106 is sewn to the inner surface 104 of the insert device 100. The bottom layer 114 is sewn along the periphery 202 and shaped in an hourglass shape, or any other suitable shape as is known in the art. Alternatively, the bottom layer 114 may also be attached to the inner surface 104 with glue, adhesive, epoxy, stitching or any other suitable fastener as is known in the art. The top layer 112 is sewn to the bottom layer 114 along the periphery 204. The top layer 112 covers the urinary source of a user and absorbs urine and moisture. The advantage of the insert device 100 is that it remains attached and secured to a user's underwear using fasteners 122 that attach to the waistband of the underwear, as well as separate fasteners 124, 126 which attach to an inner portion of the underwear.

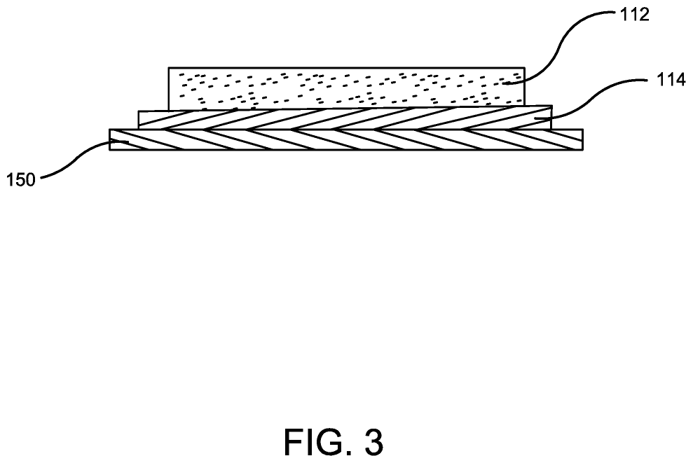

FIG. 3 illustrates a sectional view showing layers of the absorbent pad of the absorbent insert device of the present invention. The absorbent insert device 100 comprises a thin sheet of fabric (fabric body) 150 that has characteristics similar to conventional underwear fabric. The inner surface of the fabric body 150 contacts the user's body, and thereby accordingly is thin to provide comfort. On the fabric body 150, an impervious layer 114 is sewn that does not allow urine or sweat near the crotch area to penetrate and stain the underwear. The impervious layer 114 has a thickness of approximately 3 mm. On a central portion of the impervious layer 114 and in an hourglass shape, a thick absorbent layer 112 is sewn to absorb urine. The thick absorbent layer 112 is configured to absorb up to 250 ml of urine and provides protection from major bladder complications and general incontinence. The thickness of the absorbent layer 112 is approximately 6 mm, and comprises soft material. The thick absorbent layer 112 can be made up of cotton or synthetic polymers, or any other suitable material as is known in the art. The absorbent insert device 100 also controls bacterial growth and eliminates the odor of absorbed urine.

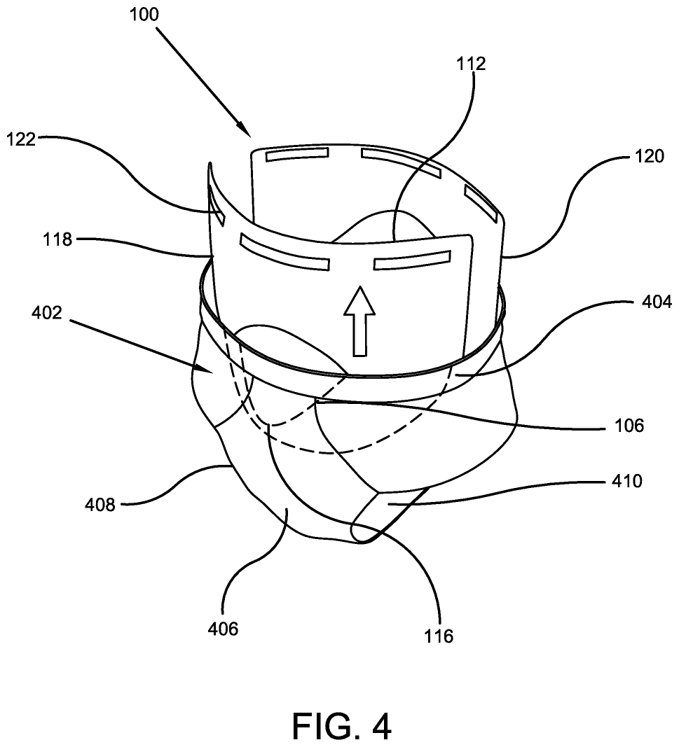

FIG. 4 illustrates a perspective view showing how the absorbent attachment device of the present invention is placed within an undergarment for use. To attach the insert device 100 to underwear 402, the insert device 100 is folded along the central bottom axis 116 such that the adhesives 122 of the front body 118 and the back body 120 are positioned along the waistband 404 of the underwear 402. The adhesives 122 can be one or more of hook and loop fasteners, Velcro®, adhesive tape, etc., or any other suitable fastening means as are known in the art to secure the attachment device 100 to the inner surface of the waistband 404. The absorbent pad 106 rests on the crotch area 406 to absorb urine leaks and to protect staining of the underwear 402.

The design and shape of the attachment device 100 conforms to the shape of the underwear 402, such that the leg openings 408, 410 of the underwear 402 are not obstructed. The absorbent pad 106 overlays the crotch area 406 of the underwear 402 and is manufactured of a material that allows the absorption of bodily fluids.

It should be appreciated that the attachment device 100 is not a pull-on type absorbent garment, but an insert type of attachment device configured to attach to the underwear 402 before the underwear 402 is worn. The attachment device 100 remains secure and stable during use and does not provide any discomfort to the user. To remove the attachment device 100 from the underwear 402, the adhesive tapes 122 are detached and the attachment device 100 can be washed and re-used.

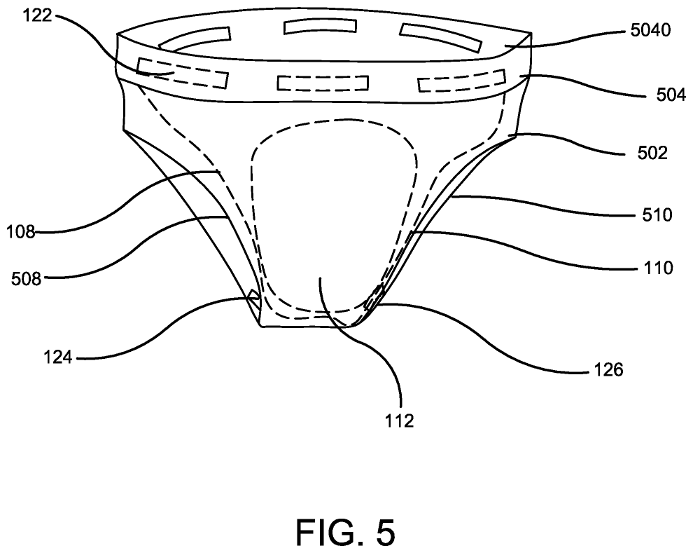

FIG. 5 illustrates a front perspective view showing an exemplary underwear with the attached absorbent underwear insert device of the present invention. As shown, the adhesive 122 of the insert device 100 is attached to inner surface 5040 of the waistband 504 to attach the insert device 100 to the underwear 502. The insert device 100 is contoured in a folded position to cover the crotch area of a user, such that the convexly opposing side 108 aligns with the side 508 and the second convexly opposing side 110 aligns with the second side 510 of the underwear 502. Additional adhesives 124, 126 allow further securement of the insert device 100 to the crotch area of the underwear 502. Further, the thick absorbent layer 112 is positioned to cover the crotch area to absorb urine and moisture and to provide additional comfort to the user.

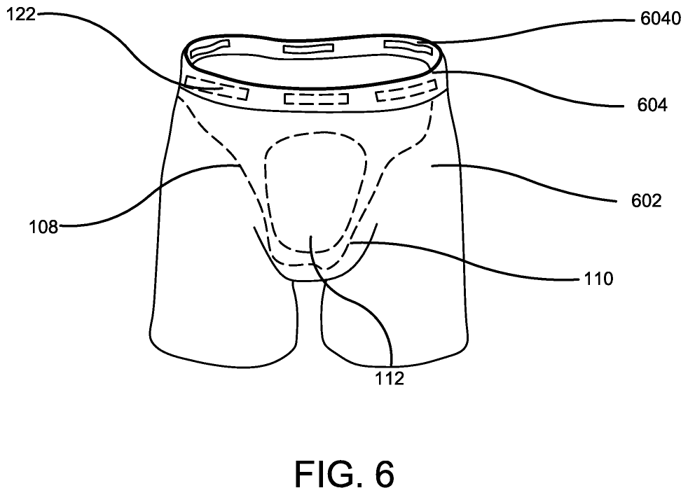

FIG. 6 illustrates a front perspective view showing trunk-shaped underwear with the attached absorbent underwear insert device of the present invention. As shown, the adhesive 122 of the insert device 100 is attached to inner surface 6040 of the waistband 604 to attach the insert device 100 to the underwear 602. The insert device 100 is contoured in a folded position to cover the crotch area of a user, such that the convexly opposing sides 108, 110 cover the crotch area of the underwear 602. The thick absorbent layer 112 is positioned to cover the crotch area to absorb urine and moisture, and to provide additional comfort to the user.

In the embodiments, the undergarment insert device 100 is constructed to provide full protection against leaks due to mild or moderate incontinence, while being comfortable, non-bulky and inconspicuous. This is achieved by using a non-bulky absorbent pad 106, and by the correct placement and size of the absorbent pad 106. The absorbent pad 106 covers the crotch portion and is disposed between the legs of the user when the undergarment insert device 100 is in use.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “absorbent underwear insert device”, “absorbent underwear attachment device”, “absorbent insert for underwear”, “urine absorbent insert device”, “absorbent pad device”, “absorbent insert device” and “insert device”, are interchangeable and refer to the absorbent underwear insert device 100 of the present invention.

Notwithstanding the forgoing, the absorbent underwear insert device 100 of the present invention can be of any suitable size and configuration as is known in the art without affecting the overall concept of the invention, provided that it accomplishes the above-stated objectives. One of ordinary skill in the art will appreciate that the size, configuration, and material of the absorbent underwear insert device 100 as shown in FIGS. 1-6 is for illustrative purposes only, and that many other sizes and shapes of the absorbent underwear insert device 100 are well within the scope of the present disclosure. Although the dimensions of the absorbent underwear insert device 100 are important design parameters for user convenience, the absorbent underwear insert device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An absorbent underwear insert device configured to be secured within a pair of underwear, the absorbent underwear insert device comprising:
a fabric body having an outer surface that adheres to a user's underwear and an inner surface configured to contact a user; and
a pair of convexly opposing sides, wherein the inner surface comprises an absorbent material pad.

2. The absorbent underwear insert device of claim 1, wherein the absorbent material pad comprises a first layer and a second layer.

3. The absorbent underwear insert device of claim 2, wherein the first layer comprises a top layer of thick absorbent material to absorb urine and sweat.

4. The absorbent underwear insert device of claim 3, wherein the second layer comprises a bottom layer of thin impervious material which prevent stains.

5. The absorbent underwear insert device of claim 1, wherein the absorbent underwear insert device is configured to fold along a central bottom fold line.

6. The absorbent underwear insert device of claim 1, wherein an outer surface near a first end and a second end comprise hook and loop fasteners.

7. The absorbent underwear insert device of claim 6, wherein the front side and the back side comprise hook and loop fasteners on the outer surface.

8. The absorbent underwear insert device of claim 7, wherein the pair of convexly opposing sides comprise hook and loop fasteners.

9. The absorbent underwear insert device of claim 1, wherein the absorbent underwear insert device is machine washable and reusable.

10. The absorbent underwear insert device of claim 1, wherein the absorbent underwear insert device is detached from a user's underwear and washed separately.

11. The absorbent underwear insert device of claim 1, wherein the absorbent underwear insert device is washed while attached to a user's underwear.

12. The absorbent underwear insert device of claim 1, wherein the absorbent material pad is attached to the fabric body via sewn seams.

13. The absorbent underwear insert device of claim 4, wherein the absorbent material pad is sewn to the inner surface of the absorbent underwear insert device.

14. The absorbent underwear insert device of claim 13, wherein the bottom layer is sewn along a periphery and shaped in an hourglass shape.

15. The absorbent underwear insert device of claim 14, wherein the top layer is sewn to the bottom layer.

16. An absorbent underwear insert device configured to be secured within a pair of underwear, the absorbent underwear insert device comprising:
a fabric body having an outer surface that adheres to a user's underwear and an inner surface configured to contact a user; and
a pair of convexly opposing sides, wherein the inner surface comprises an absorbent material pad, and further wherein the absorbent material pad comprises a top layer of thick absorbent material to absorb urine and sweat and a bottom layer of thin impervious material which prevent stains, and further wherein the outer surface near a first end and a second end comprises hook and loop fasteners.

17. The absorbent underwear insert device of claim 16, wherein the pair of convexly opposing sides comprise hook and loop fasteners.

18. The absorbent underwear insert device of claim 17, wherein the absorbent material pad is attached to the fabric body via sewn seams.

19. An absorbent underwear insert device configured to be secured within a pair of underwear, the absorbent underwear insert device comprising:
a fabric body having an outer surface that adheres to a user's underwear and an inner surface configured to contact a user; and
a pair of convexly opposing sides; and
wherein the inner surface comprises an absorbent material pad; and
wherein the absorbent material pad comprises a top layer of thick absorbent material to absorb urine and sweat and a bottom layer of thin impervious material which prevent stains; and
wherein the outer surface near a first end and a second end comprises hook and loop fasteners; and
wherein the absorbent material pad is sewn to the inner surface; and
further wherein the bottom layer is sewn along a periphery and the top layer is sewn to the bottom layer.

20. The absorbent underwear insert device of claim 19, wherein the absorbent underwear insert device is machine washable and reusable.
